(19) United States
(12) Patent Application Publication
(10) Pub. No.: US 2018/0085025 A1
(43) Pub. Date: Mar. 29, 2018

(54) MAGNETIC RESONANCE APPARATUS AND OPERATING METHOD WITH DIFFERENT TABLE DISPLACEMENT MODES

(71) Applicant: Siemens Healthcare GmbH, Erlangen (DE)

(72) Inventors: Johannes Ehrl, Pinzberg (DE); Kirstin Jattke, Nuernberg (DE); Miriam Keil, Erlangen-Dechsendorf (DE); Christof Krellmann, Erlangen (DE); Stephan Nufer, Erlangen (DE); Peter Schmitt, Weisendorf (DE)

(73) Assignee: Siemens Healthcare GmbH, Erlangen (DE)

(21) Appl. No.: 15/712,982

(22) Filed: Sep. 22, 2017

(30) Foreign Application Priority Data
Sep. 23, 2016 (DE) 102016218356.7

(51) Int. Cl. A61B 5/055; A61B 6/00; A61B 5/00; A61B 6/04; G01R 33/48

(52) U.S. Cl. CPC A61B 5/055; A61B 6/488; G06T 2207/30004; A61B 6/04; G01R 33/48; A61B 5/7271

(57) ABSTRACT

In a magnetic resonance system and an operating method therefor, wherein measurement data are able to be recorded in a first recording mode without displacement of a patient table or in a second recording mode with displacement of a patient table before measurement begins, an image dataset is provided to a computer, which represents an image of at least a part of an examination volume, and the image dataset is displayed together with at least one setting facilitator for setting at least one measurement parameter of a measurement dataset to be recorded. An information symbol about the recording mode is superimposed on the image dataset, at least for a time.

### BACKGROUND OF THE INVENTION

### Field of the Invention

The present invention concerns a method for operating a magnetic resonance apparatus, wherein measurement data are able to be in a recording mode without displacement of a patient table or in a recording mode with displacement of the patient table before the beginning of the measurement.

### Description of the Prior Art

Usually measurements for magnetic resonance systems are planned by an overview or planning image of the region of the patient to be examined being displayed. After selection of a measurement sequence, a graphical element for slice planning is superimposed on the displayed image. After positioning of the slices or the parameterization of slice thickness, as well as designating further recording parameters, the measurement can be started.

If the slice in this case is in the homogeneous magnetic field region around the isocenter of the data acquisition scanner, the measurement can be started directly. Otherwise, the patient table is moved, so that the middle of the slice is, in the longitudinal direction of the patient table, at the location of the isocenter. Subsequently adjustment measurements are undertaken once more, such as shimming and determination of the resonant frequency, and thereafter the measurement is started.

Of course a number of measurements can also be planned in advance. These are then processed one after another and the table movement along with all necessary adjustment measurements are then carried out as needed.

A problem with this method of operation is that many table movements and adjustment measurements are needed. This leads to a lengthening of the measurement time, since the adjustment measurements take time. The comfort of the patient and the operating efficiency of the magnetic resonance system suffer as a result.

### SUMMARY OF THE INVENTION

An object of the present invention is therefore to further develop a method of the type described at the outset so that an improved planning of magnetic resonance measurements is made possible.

This object is achieved in accordance with the invention by a method for operating a magnetic resonance apparatus, wherein measurement data are to be recorded with the scanner of the magnetic resonance apparatus in a first recording mode without displacement of a patient table or in a second recording mode with displacement of a patient table before the measurement begins, with the steps of providing an image dataset to a computer, which represents an image of at least a portion of an examination volume of a subject, and at a display in communication with the computer, displaying the image dataset together with at least one setting facilitator for setting at least one measurement parameter of a measurement dataset, and superimposing on the image dataset, at least for a time, of an information symbol about the current recording mode.

The basis of the invention is that the apparatus operator, even during planning of a measurement, receives information about whether or not the parameters that the operator has chosen make a movement of the table necessary before measurement begins. If possible a measurement can be planned and optimized so that no patient table movement or no displacement of the patient table is necessary.

This enables savings or reductions to be made in the adjustment measurements that are then necessary, through which the time that patients spend in the magnetic resonance system is shortened. To a lesser extent the SAR can also be lowered.

The image dataset provided is also called an overview dataset or a scout scan or the like. This usually involves a dataset recorded with a fast imaging method such as TrueFISP or FLASH. It can be composed of one or more individual images, e.g. three images with recording planes at right angles to one another. These are aligned in the sagittal, coronal and transverse planes. The image dataset can also be already-scanned 3D or 2D images.

The image dataset is used for planning of the subsequent measurements. Therefore selection items and/or setting items for selection and setting of measurements can also be displayed at the same time as the image dataset.

Naturally the display is undertaken at a display monitor. The setting facilitator is usually a region shown on the display monitor. In such cases this can be a slider control, an input field, a button, a selection list, a marking field, a marking point or any other commonly employed setting facilitator. The setting facilitator is able to be operated via the keyboard or the mouse.

In particular the setting facilitator can be a setting item that sets the slice position and/or the slice thickness. This setting item can be embodied as a rectangular border or as a frame, which is able to be moved freely over the image dataset and is superimposed thereon.

Preferably the setting facilitator is for the slice position. It becomes necessary to move the patient table if the slice lies outside the homogeneous region. While with most known magnetic resonance scanners each displacement of the middle of the selected slice leads to a table movement, this is now only the case when the setting departs from a predetermined measurement volume.

The fact that the information symbol about the recording mode is displayed at the same time as the setting facilitator enables the user to see, when setting the parameters of a measurement, whether the selected measurement parameters lead to a displacement of the table or not. Thus unnecessary table movements and subsequent adjustment measurements can be avoided.

Preferably position information about the patient table, particularly as a function of the position of the selected slice, can be displayed as the information symbol or as additional information.

The position information can likewise be displayed just for a time. A displacement in relation to the middle of the image of the image dataset can in such cases show a table movement. Thus the display of the table position is then also a display of the recording mode.

As an alternative, the information symbol and the position information can be displayed independently of one another or at the same time. In this case the information that a patient table movement is taking place is shown twice and thus in a more easily recognizable way.

Preferably the information symbol and/or the position information can be displayed during the activation of the setting facilitator. Preferably the setting facilitator is for setting a displacement-relevant parameter, for example the slice position and/or size.

If the information symbol and/or the position information are displayed just for a time, the information should preferably be displayed when a setting facilitator that can result in a patient table movement is operated. The operator can then ensure during the interaction that the operator proceeds carefully during the setting or optimizes matters, to the extent that no table displacement becomes necessary.

As an alternative, the information symbol and/or the position information can be displayed during the activation of the setting facilitator and for a predetermined period of time after the activation. By contrast with the first alternative, the display also persists for some time, so that the user has feedback, even without having to keep the setting facilitator activated.

As a further alternative, the information symbol and/or the position information can be superimposed onto the image dataset for the entire duration of the display. This does mean that a warning function is lost, but it can be sufficient for a displayed information symbol to be changed in order to warn the user.

As a further alternative, the information symbol and/or the position information can be superimposed on the image dataset, if the consequence of a change to a parameter by interaction with the setting facilitator is a table movement.

In addition or as an alternative, in the period or at the point in time for displaying the information symbol and/or the position information, the display of the information symbol and/or of the position information can be changed, if the consequence of changing a parameter by use of the setting facilitator is a table movement. Then, for example, not only is the position of the position information changed, but also the presentation, for example the color.

In such cases the display variants can also be combined. Preferably the information symbol continues to be displayed while the image dataset is being displayed. When there is a change of recording mode the display is changed.

However, the position information of the patient table is only displayed when the consequence of a setting, of the slice for example, is a table movement. On the basis of this display the user can recognize whether the table movement is considerable or there is just a slight movement. With small table movements it is worth attempting to modify the measurement parameters in order to avoid the table movement entirely.

It should be noted that changing the recording mode from the first to the second recording mode is synonymous with a table movement occurring.

Preferably the middle of the patient table in the longitudinal direction can be displayed as the position information. In this case the middle can be shown as a line. This type of display allows the user to recognize immediately by how much the patient table is being moved in its longitudinal direction.

Preferably the information symbol about the recording mode can be displayed at the edge of the image dataset. At this point the examination object is usually not covered.

The information symbol can have the same outer contour for both recording modes. This means that it can still be recognized that a symbol is shown for the recording mode, even if this changes.

The described steps, except for the setting of a few measurement parameters, are performed in this case in an automated manner by a control facilitator. In particular the display of the information symbol and/or of the position information is performed in an automated manner by the control computer.

The above object is also achieved by a control computer for a magnetic resonance apparatus that is configured (programmed) to implement the inventive method as described.

The present invention also encompasses a non-transitory, computer-readable data storage medium encoded with programming instructions (program code) that, when the storage medium is loaded into a control computer or computer system of a magnetic resonance apparatus, cause the computer or computer system to operate the magnetic resonance apparatus in order to implement any or all of the embodiments of the method according to the invention, as described above.

The object underlying the present invention is also achieved by a magnetic resonance apparatus with a control computer that operates the apparatus in order to implement the inventive method as described.

Embodiments of the inventive data storage medium and the inventive magnetic resonance apparatus correspond to the embodiments of the inventive method as described above.

The aforementioned method can be implemented in the control computer as software, or as (hard-wired) hardware.

### BRIEF DESCRIPTION OF THE DRAWINGS

FIG. 1 schematically illustrates a magnetic resonance system constructed and operating in accordance with the invention.

FIG. 2 shows a monitor display in a first embodiment of the invention.

FIG. 3 shows a monitor display in a second embodiment of the invention.

FIG. 4 shows a monitor display in a third embodiment of the invention.

FIG. 5 shows a monitor display in a fourth embodiment of the invention.

### DESCRIPTION OF THE PREFERRED EMBODIMENTS

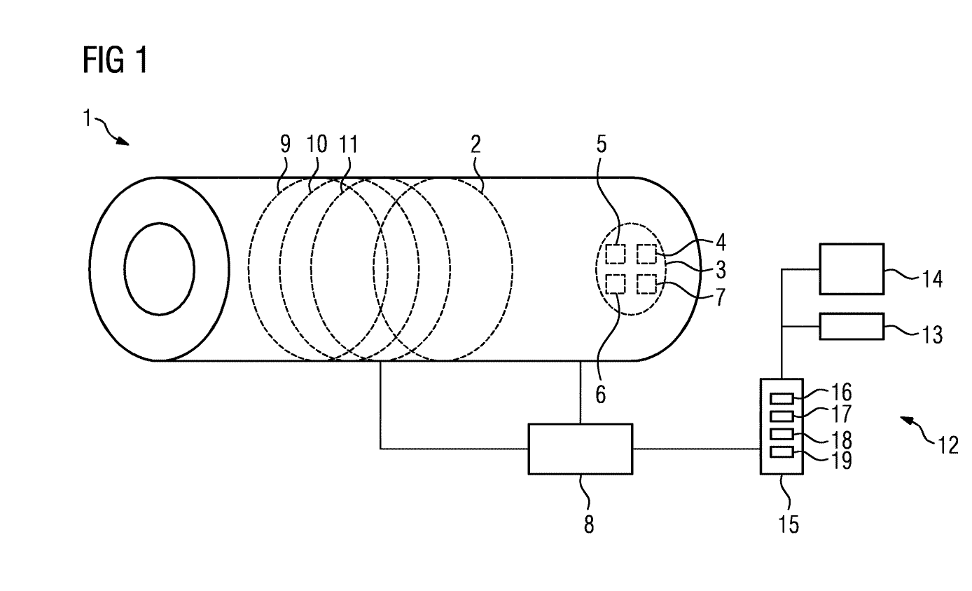

FIG. 1 illustrates a magnetic resonance system 1. As well as a radio-frequency coil 2, embodied as a body coil, the scanner has a coil array 3 with coils 4, 5, 6 and 7 as well as a control computer 8. The coil 2 is used as an excitation coil for exciting the magnetization.

The coil array 3 is used as a detector coil for reading out the measurement signal. In this case the coils 4, 5, 6 and 7 of the coil array 3 read out the measurement signal at the same time.

Instead of a coil array 3, an individual coil can be used as a detection coil. A single coil or a single coil array can also be used as an excitation coil and detection coil.

The gradient coils 9, 10 and 11 are needed for imaging and are therefore present in every MR tomography device. The gradient coils 9, 10 and 11 create gradient fields in three directions. These are superimposed to create the gradients used in a recording sequence, which are present in the read, phase and slice direction. This means that the gradients used in a sequence, depending on their location, are composed of the gradients individually or in any given combination.

The gradient coils 9, 10 and 11 or the fields created with them are known to be needed for spatially encoding the MR signals. Through the repeated variation of at least one value for powering one of the gradient coils 9, 10 and 11 a phase encoding is created.

Furthermore, the magnetic resonance system 1 has a computer console 12. This has a keyboard 13 as its input unit, a monitor 14 as its display unit and a computer 15 with processor 16, motherboard 17, main memory 18, data medium 19 and the further usual components of a computer as its computer hardware. In this case the parts of the software that are currently needed for carrying out the work on the computer 15 are loaded into the main memory 18 from the data storage medium 19.

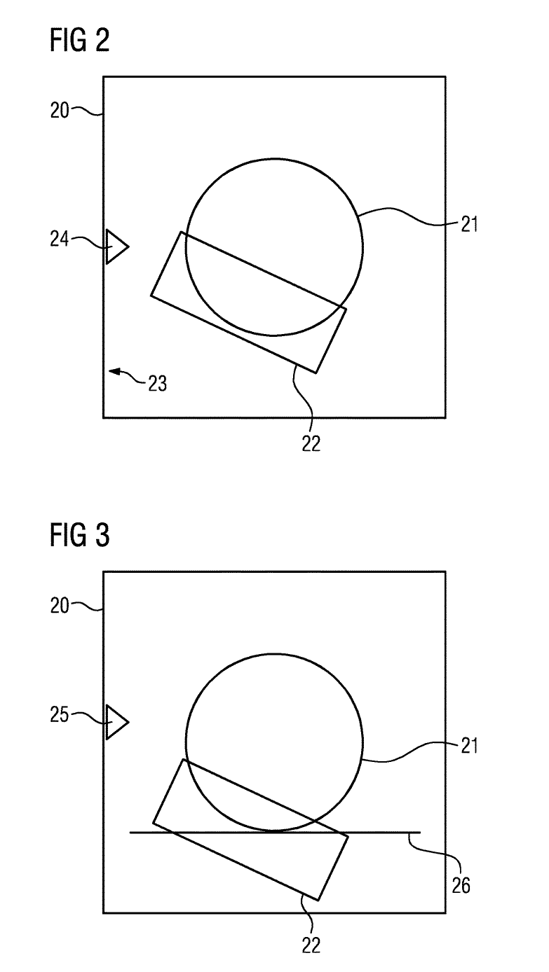

FIG. 2 shows an example of a display on the monitor 14 with an image 20 of an image dataset, in which a head 21 is displayed as the examination region in sections in the sagittal or coronal direction. The image 20 and, if necessary, the further images of the image dataset have been recorded for positioning of subsequent measurements. Therefore the image 20 has a frame 22 superimposed thereon, as a setting facilitator for defining the slice position.

Other setting facilitators can also be present as well as the frame 22 on the monitor 14, for example a setting facilitator for defining the number of slices to be recorded, a setting facilitator for selecting the measurement sequence, etc.

The user can change the position of the slice to be recorded by clicking on the frame 22 and activating it by doing so. The frame 22 is then movable, either by the mouse being moved with a button pressed, or by a movement being controlled via the arrow keys for example.

The computer 15 then converts the movement of the frame 22 into the corresponding measurement parameters. In this case the frame 22 shows the location of the slice to be recorded, which is therefore also referred to as the slice being moved.

With small movements of the frame 22 and thus of the slice to be recorded, the measurement slice remains within a predetermined, homogeneous volume and the measurement can be made without new adjustment measurements being necessary. Therefore an information symbol 24 is displayed at the edge 23, which shows that a first recording mode without movement of a patient table is being used for the recording of the measurement.

Regardless of the further features in relation to the image 20 the information symbol 24 is preferably embodied as a triangle or features a triangle. Also preferably a tip of the triangle can display a preferred position of the patient table. In this case a reference position can be involved, for example the position of the patient table at the beginning of the measurement, or the middle of the isocenter. The triangle can in such cases also adjoin a semicircle or the like. The center of the image can be marked by means of the tip.

This is of advantage when, as shown in FIG. 3, the frame 22 is displaced so far that, in the recording of the measurement dataset that is currently being prepared, a displacement of the patient table becomes necessary. The information symbol 25 can then be displayed. The use of the information symbol 24 or 25 is thus needed for displaying the recording mode.

At the same time as the display of the information symbol 24 and/or 25, a line 26 can also be displayed as position information about the middle of the patient table. In this case the embodiment of the information symbols 24 and 25 as a triangle or with a triangle is particularly advantageous, because here the distance between the reference position, and thereby between the isocenter of the magnetic resonance system 1 and the line 26 and thus the potential future middle of the table is able to be acquired easily and intuitively.

With the distance shown in FIG. 3 it is not possible to avoid a table movement and undertaking adjustment measurements through an optimization of the measurement parameters and thereby of the slice position.

The line 26 is only displayed for the embodiments according to FIGS. 2 and 3 when the recording mode is changed and thus a table movement takes place.

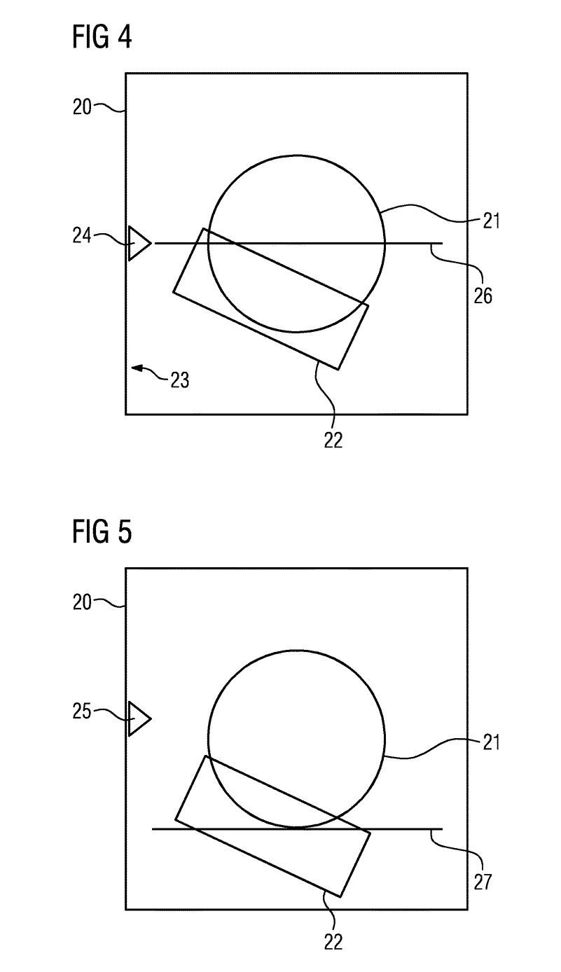

FIG. 4 shows an alternate embodiment to FIG. 2. In this embodiment, the line 26 is always displayed as position information, even if the information symbol 24 shows the first recording mode without table movement.

In this embodiment, the user is shown via the change of the information symbols 24 and 25, as well as a movement of the line 26, when the measurement parameters require a patient table movement.

To enhance perceptibility, as shown in FIG. 5, the line 26 can be changed into the line 27 when the recording mode is switched from the first into the second recording mode. The line 27 can have a different color from the line 26 and/or it can flash or be distinguished from the line 26 in some other way.

These variations can basically also be used for the embodiment according to FIG. 2, however here an additional distinction is actually not necessary.

Thus the user can avoid unnecessary patient table movements during planning or can avoid a change of recording mode.

After definition of all measurements, the execution sequence can also be optimized such that, although all necessary table movements are carried out, such movements are minimized. For example, starting from the current position, all table movements can be put into a contiguous area after one another, so that no table movements are necessary within these measurements. It can also be displayed to the user after such an optimization where table movements are necessary at the moment but would be avoidable with parameter optimization.

Although modifications and changes may be suggested by those skilled in the art, it is the intention of the Applicant to embody within the patent warranted hereon all changes and modifications as reasonably and properly come within the scope of the Applicant's contribution to the art.

1. A method for operating a magnetic resonance system (1), wherein measurement data is able to be recorded with the magnetic resonance system (1) in a first recording mode without displacement of a patient table or in a second recording mode with displacement of a patient table before measurement begins, comprising:
providing an image dataset to a computer, said image data set comprising at least a part of an image of an examination volume;
at a display in communication with said computer, displaying the image dataset together with at least one setting facilitator in order to set at least one measurement parameter of a measurement dataset to be recorded; and
at said display, superimposing, at least for a time, an information symbol about the recording mode on the image dataset.

2. The method as claimed in claim 1, comprising displaying, as said setting facilitator, a setting facilitator for slice selection.

3. The method as claimed in claim 2, comprising displaying position information about the patient table as a function of the position of the selected slice, as the information symbol or as additional information.

4. The method as claimed in claim 3, comprising displaying at least one of the information symbol or the position information during activation of the setting facilitator.

5. The method as claimed in claim 3, comprising displaying at least one of the information symbol or the position information during activation of the setting facilitator and for a predetermined period of time after said activation.

6. The method as claimed in claim 3, comprising displaying at least one of the information symbol or the position information superimposed on the image dataset for an entire duration of the display.

7. The method as claimed in claim 3, comprising displaying the middle of the patient table in a longitudinal direction as said position information.

8. The method as claimed in claim 7, comprising displaying the middle of the patient table as a line.

9. The method as claimed in claim 1, comprising displaying the information symbol about the recording mode at an edge of the image dataset.

10. The method as claimed in claim 1, comprising using an information symbol for both recording modes that has the same outer contour for each recording mode.

11. A non-transitory, computer-readable data storage medium encoded with programming instructions, said storage medium being loaded into a computer system of a magnetic resonance apparatus comprising a movable patient table with which measurement data are able to be acquired in a first recording mode without a displacement of the patient table or in a second recording mode with displacement of the patient table before a measurement begins, said programming instructions causing said computer system to:
receive an image dataset comprising at least a part of an image of an examination volume;
at a display in communication with said computer system, display the image dataset together with at least one setting facilitator in order to set at least one measurement parameter of a measurement dataset to be recorded; and
at said display, superimpose, at least for a time, an information symbol about the recording mode on the image dataset.

12. A magnetic resonance apparatus comprising:
a magnetic resonance data acquisition scanner comprising a movable patient table;
a computer configured to operate said MR data acquisition scanner in a first recording mode without displacement of the patient table or in a second recording mode with displacement of the patient table;
said computer being configured to receive an image dataset comprising at least a part of an image of an examination volume;
a display in communication with said computer;
said computer being configured to display the image dataset together with at least one setting facilitator at said display, in order to set at least one measurement parameter of a measurement dataset to be recorded; and
said computer being configured to superimpose, at said display at least for a time, an information symbol about the recording mode on the image dataset.
